(19) United States
(12) Patent Application Publication
(10) Pub. No.: US 2013/0302873 A1
(43) Pub. Date: Nov. 14, 2013

(54) Methods of Making Material Coatings for Self-cleaning and Self-decontamination of Metal Surfaces

(71) Applicants: Alok Singh, Springfield, VA (US); Walter J. Dressick, Waldorf, MD (US)

(72) Inventors: Alok Singh, Springfield, VA (US); Walter J. Dressick, Waldorf, MD (US)

(21) Appl. No.: 13/706,651

(22) Filed: Dec. 6, 2012

Related U.S. Application Data
(62) Division of application No. 11/907,197, filed on Oct. 10, 2007, now abandoned.

(51) Int. Cl. A61L 2/238

(52) U.S. Cl. CPC A61L 2/238
USPC 435/174; 422/8

(57) ABSTRACT

A method of making a composite structure exhibiting the ability to degrade chemical or biological agents upon contact comprising a substrate to be protected from the deleterious effects of chemical or biological agents possessing surface groups capable of deactivating materials having the ability to degrade chemical or biological agents, a buffer film, coated onto the substrate, that blocks the ability of the substrate surface groups to deactivate the materials having the ability to degrade chemical or biological agents, and a protective film, coated onto the buffer film, containing materials having the ability to degrade chemical or biological agents encapsulated in or comprising the outer surface of the protective film.

This application is a divisional application of and claims the benefits of U.S. patent application Ser. No. 11/907,197 filed Oct. 10, 2007, which claimed the benefits of provisional application No. 60/851,074 filed on Oct. 12, 2006, both of which are herein incorporated by reference.

In today's society, there is a constant and growing need to protect persons from exposure to microbial and chemical threats to health and well-being. In particular, the continuous mutations of microbial life forms invariably result in adaptations leading to development of resistance to drugs used to control their populations. For example, many diseases, such as tuberculosis, gonorrhea, malaria, and childhood ear infections, that were once cured or controlled have become sufficiently drug resistant that they represent serious new health threats. In fact, about 70 percent of bacteria that cause infections in hospitals are currently resistant to at least one of the drugs most commonly used to treat infections. For example, Methicillin-resistant Staphylococcus aureus (MRSA, i.e., flesh-eating bacteria) is now a major problem around the world, causing hospital-acquired infections as well as infections in the community (H. F. Chambers, Emerg. Infect. Diseases 2001, 7, 178; B. C. Herold, et. al., JAMA 1998, 279, 593). Even more troubling is the development of new and deadlier microbial strains, such as Clostridium difficile, an organism associated with severe and potentially fatal intestinal distress, that exhibit resistance to antibiotic treatments and pose an increasingly serious health problem for hospitals (L. C. McDonald, et. al., N. Engl. J. Med. 2005, 353, 1503). Similar concerns trouble the food industry, where microbial contamination of food through contact with contaminated food storage containers and preparation surfaces (e.g., counter tops) can lead to severe health problems for consumers. Each year several million people in the United States are infected with E. Coli, Salmonella, and Campylobacter, which usually cause severe gastrointestinal distress (e.g., diarrhea). Salmonella infections are typically treated with trimethoprim-sulfamethoxazole, ampicillin, fluoroquinolones or third-generation cephalosporins. However, some Salmonella and Campylobacter infections have now become resistant to these drugs.

Likewise, the explosive growth of new materials (i.e., chemicals) that accompanies industrial and technological progress in our society provides a myriad of potential new exposure health threats to our populace. Such threats may not be immediately apparent in many cases until the damage has become sufficiently severe and health is compromised to the point that distinct symptoms appear. For example, for many chemicals, such as pesticides, low exposure levels over a long period of time can lead to such cumulative damage. In other cases, such as an accidental chemical spill or a targeted chemical release associated with an act of sabotage or terror ism, the health effects are more immediate. With regard to the latter, similar arguments can be made for accidental or deliberate release of microbial life forms. However, in these cases, the deleterious health effects on the exposed populace will usually become apparent only after a sufficient incubation period.

No matter what the material, chemical or biological, and mode of exposure, deliberate or accidental, exposure has both immediate and prolonged consequences for the populace. The immediate consequences, i.e., development of health issues for the exposed persons manifested by symptoms associated with chemical toxicity or microbial infection (i.e., disease), are readily apparent. While these are serious issues, perhaps a more insidious problem is the long-term effects. For example, identification of the source of the contamination is critically important so that remedial measures can quickly be taken to prevent further injury due to chemical exposure or microbial infection to the populace. Once identified, remediation of the contaminated or infected areas can prove difficult and expensive in terms of the time, manpower, and financial resources required to adequately address the problem.

Consequently, there is a clear need to develop measures that can successfully address such contamination issues rapidly and completely in an economical manner. One promising means for doing so involves the development of self-cleaning or self-decontaminating surfaces. Upon contact with a chemically or biologically hazardous substance, such materials are capable of catalytically degrading said hazardous substance to less toxic or, ideally, non-toxic substances. In this manner, the hazardous substance is continually destroyed by contact with the self-cleaning or self-decontaminating surface. Because no hazardous material accumulates, there is no need to clean (i.e., decontaminate) these self-cleaning or self-decontaminating materials via conventional means, e.g., through treatment with an aqueous soap solution to remove pesticide residue or aqueous bleach or alcohol solution to kill adsorbed bacteria.

One type of said self-cleaning or self-decontaminating materials useful for catalytic degradation of chemical toxins, such as organophosphorous pesticides and nerve agents, generally comprises a polyelectrolyte multilayer film containing organophosphorous hydrolases and related enzymes, as described in the following publications, the contents of which are incorporated herein by reference in their entirety (Y. Lee, et. al., Langmuir 2003, 19, 1330; A. Singh, et. al., Adv. Mater. 2004, 16, 2112; A. Singh, W. J. Dressick, and Y. Lee, “Catalytic Enzyme-modified Textiles for Active Protection From Toxins”, U.S. Pat. No. 7,270,973 (filed 20 May 2004 and issued 18 Sep. 2007); A. Singh, Y. Lee, I. Stanish, E. Chang, and W. J. Dressick, “Catalytic Surfaces for Active Protection From Toxins”, U.S. Pat. No. 7,067,294 (filed 23 Dec. 2003 and issued 27 Jun. 2006)). Said films are typically fabricated using a layer-by-layer approach (G. Decher, Science 1997, 277, 1232) on fabrics (for manufacture of protective clothing) or polymer beads (for manufacture of protective filters). The films are conveniently assembled by exploiting electrostatic attractions between the charged surface groups of the enzymes and oppositely-charged polyelectrolytes via alternate dipcoating of the substrate (i.e., fabric or beads) in separate aqueous solutions containing the enzymes and polyelectrolytes. Upon contact with a solution containing methylparathion, a pesticide, fabrics or beads coated with these self-decontaminating multilayer-enzyme coatings efficiently hydrolyze the methylparathion (MPT) to less toxic p-nitrophenol (PNP) and O,O-dimethylphosphorothioxo-1-ol products (A. Singh, et. al., Adv. Mater. 2004, 16, 2112).

Similar multilayer films having antimicrobial properties have also been described. For example, multilayers can be formed via alternating assembly of polyacrylates (PAA) and polyallylamine hydrochloride (PAH) in solutions at ˜2.5<pH<˜4.5. Under these conditions, a fraction of the carboxylic acid (i.e., COOH) groups of the PAA remain protonated and are unable to electrostatically bind protonated amine groups of the PAH. Upon treatment of the resulting multilayer film with solutions containing ionic silver salts, Ag⁺ ions can permeate the film and bind to these available carboxylic acid sites via displacement of H⁺ from the COOH groups. Subsequent addition of a reducing agent, such as sodium borohydride or dimethylamine borane leads to reduction of the bound Ag⁺ to silver atoms, which aggregate to form Ag(0) nanoparticles entrapped within the multilayer film (T. C. Wang, et. al., Langmuir 2002, 18, 3370). Composite multilayer-Ag(0) films of these sorts exhibit antibacterial properties, which have been attributed to slow oxidation and dissolution of the Ag(0) within the film to generate Ag⁺ ions that diffuse out of the film. These released Ag⁺ ions efficiently kill bacteria adsorbed to the film surface (D. Lee, et. al., Langmuir 2005, 21, 9651). Other metals, such as Cu, also possess biocidal properties (N. Cioffi, et. al., Chem. Mater. 2005, 21, 5255) and their nanoparticles have also been shown to function efficiently as components of antimicrobial surfaces in polymer composites.

Alternate means to fabricate antimicrobial surfaces involve direct grafting of a passive or active antimicrobial agent to the surface of the desired substrate. Passive agents include various organic salts, such as quaternary ammonium (L. P. Sun, et. al., Polymer 2006, 47, 1796), quaternary phosphonium (A. Kanazawa, et. al., J. Appl. Polym. Sci. 1994, 54, 1305), and alkylpyridinium salts (F. X. Hu, et. al., Biotechnol. Bioeng. 2005, 89, 474). These materials typically possess one or more n-alkyl chains chemically bound to their cationic N (or P) heteroatom. They are thought to kill microbial cells through a lysing mechanism involving: (1) direct penetration of the n-alkyl chain into and disruption of the bilayer comprising the microbial cell wall (S. B. Lee, et. al., Biomacromolecules 2004, 5, 877) as shown in FIG. 1, and/or; (2) displacement of Ca²⁺ and Mg²⁺ ions in the microbial cell wall that function as the “glue” maintaining the cell wall integrity, again leading to leakage of the cell contents and cell death (R. Kügler, et. al., Microbiology 2005, 151, 1341). In support of these mechanisms, n-alkyl chains of as few as 2-4 carbons appear capable of lysing microbial cells, with the greatest killing efficiencies typically noted for n-alkyl chains of 12-16 carbon atoms in length (i.e., of similar size to the lipids comprising the cell walls). Surface concentrations of these organic salts (e.g., number of quaternary amine or pyridinium sites per square centimeter of substrate surface, N⁺/cm²) required to kill microbes depend upon a variety of factors, such as the organic salt used and the type microbe and its metabolic state. For example, S. epidermis (R. Kügler, et. al., Microbiology 2005, 151, 1341)

in its growth phase is instantly killed on a surface bearing polyvinyl(N-butyl-pyridinium) groups at a density ≧˜10¹³ N⁺/cm². In contrast, in its quiescent phase death occurs instantly only on surfaces having a pyridinium group density ≧˜10¹⁴ N⁺/cm². Although quiescent S. epidermis can be killed on surfaces having pyridinium group densities ≦˜10¹⁴ N⁺/cm², death occurs only after more prolonged contact, i.e., survival times as long as ˜2 hr are noted. For E. coli, death occurs instantly at pyridinium surface densities ≧˜10¹² N⁺/cm² in its growth phase and ≧˜10¹⁴ N⁺/cm² in its quiescent state. Similar results have been obtained in other studies using E. Coli and surfaces bearing polyvinyl(N-hexyl-pyridinium) groups (L. Cen, et. al., Langmuir 2003, 19, 10295).

An active agent for the destruction of microbes releases a chemical species from the protected surface, usually but not always on contact of the surface by the microbe, to attack and kill the microbe. For example, organic quaternary ammonium salts attached to a surface via a weak ester linkage have been demonstrated as active agents for the destruction of microbes; hydrolysis of the ester by the microbe releases the quaternary ammonium salt into the environment, where its interaction with the lipid bilayer of the cell wall leads to microbe death (P. J. McCubbin, et. al., J. Appl. Polym. Sci. 2006, 100, 538). However, most active agents comprise more conventional chemical species, such as hypochlorites (i.e., bleach). In particular, melamine derivatives, such as the 2-amino-4-chloro-6-hydroxy-S-triazine (ACHT) species shown in FIG. 2, form chloromelamine derivatives via chlorination of the amine group in the presence of bleach (Y. Sun, et. al., Ind. Eng. Chem. Res. 2005, 44, 7916). Chloromelamine groups are particularly effective agents for the destruction of both gram positive and gram negative bacteria via release of active chlorine upon contact with bacteria for both water-borne and air-borne surface contamination modes. ACHT is readily grafted to cellulose (i.e., fabric) surfaces via reaction of its hydroxyl site to produce protected surfaces that maintain the durability or the original cellulose substrate (M. Braun, et. al., J. Polym. Sci. A—Polym. Chem. 2004, 42, 3818). In addition, because chlorination is a reversible reaction, surfaces treated with ACHT can be easily recharged by rinsing with a bleach solution to regenerate antimicrobial activity.

Metals comprise an important aspect of the infrastructure of our society. Aluminum, in particular, is widely used for a variety of applications critical to modern life due to its favorable chemical and physical properties, including its high electrical and thermal conductivity, good reflectivity, resistance to corrosion, and strength and light weight. For example, its good strength and light weight makes aluminum metal a primary component of airplane frames and bodies, as well as surgical instruments. Because of its high electrical and thermal conductivities, aluminum metal remains a principle component in the fabrication of electrical power lines and electrical interconnects comprising power distribution modes in integrated circuits. Likewise, aluminum's high reflectivity and resistance to corrosion make it a preferred choice for optical applications, as well as the fabrication of countertops, kitchen appliances, and as a decorative metal for items such as handrails and elevator panels.

Unfortunately, the adaptation of the technologies described herein thus far for the protection of aluminum and other metals is not straightforward. Specifically, the surface chemical and physical properties of metals can influence the activity and function of such self-cleaning or self-decontaminating protective films. For example, aluminum metal is protected by a thin layer of aluminum oxide (i.e., alumina) strongly chemisorbed to the metal surface. The structure of this oxide, including the density of hydroxyl groups and degree of hydration, can influence surface properties of the material, as can surface treatments. For example, hydroxyl groups surface densities can be decreased by thermal treatments, affecting the acidity of the hydroxyl sites as shown by the rather large range of isoelectric points (i.e., ˜5.0<pI<˜9.4) measured for different forms of the oxide (G. V. Franks, et. al., Coll. Surf A 2003, 214, 99). This ability to chemically treat alumina to produce acidic, neutral, or basic surface species forms the basis for alumina chromatography. However, it can also adversely affect the function of protective coatings. For example, it is well-known that adsorption of active enzymes directly to alumina or other metal oxide surfaces can reduce or eliminate enzyme activity due to denaturation, unless steps are taken to carefully control the surface morphology/structure and chemical composition (see, e.g.; W. Tischer, et. al., Topics Curr. Chem. 1999, 200, 95; L. Gianfreda, et. al., Molec. Cellular Biochem. 1991, 100, 97; A. Mueller, Mini Rev. Med. Chem. 2005, 5, 231). Unfortunately, conditions required for optimal enzyme adsorption and function at such oxide surfaces may compromise other functions, such as surface conductivity or reflectivity, critically important for the intended application of the material. Consequently, while the metal may acquire self-cleaning or self-decontaminating properties, the loss of these other desirable traits may render it useless for the desired application.

Likewise, the environment at the alumina and other oxide surfaces can also influence efforts to graft molecular materials, such as ACHT and related molecules, having useful antimicrobial activity. For example, aminopropylsiloxane self-assembled monolayers (SAMs) are readily chemisorbed to alumina, silica, and other oxide surfaces (Chen, et. al., J. Electrochem. Soc. 1999, 146, 1421). The alkylamine functional group in the resulting SAM chemisorbed on fused silica slides is readily reacted by stirring a cyanuric chloride (FIG. 3) solution in chloroform for ˜1 week at room temperature. The alkylamine displaces one of the cyanuric chloride Cl groups to form a surface-bound 2-aminopropyl-4,6-dichloro-S-triazine material on the fused silica. FIG. 4 shows the presence of a strong UV absorbance band at λ<200 nm with a shoulder at λ˜320 nm indicating the formation of the surface-bound 2-aminopropyl-4,6-dichloro-S-triazine material on the fused silica. In principle, one can react the remaining two Cl groups to form ACHT-like materials on the surface. However, in practice, the ability to form and retain a surface-bound product is not always straightforward. For example, treatment with a DMF solution of 4-N-methylaminoethylpyridine at 60° C. for 6 hours leads to complete removal of the triazine residue from the surface, rather than addition of the N-methylaminoethylpyridine to the chemisorbed 2-aminopropyl-4,6-dichloro-S-triazine material on the fused silica. In contrast, reaction with the hydroxyl group of β-cyclodextrin under similar conditions effectively displaces a Cl from the chemisorbed 2-aminopropyl-4,6-dichloro-S-triazine, creating a hybrid 2-aminopropyl-4-β-cyclodextrin-6-chloro-S-triazine material (the hydroxyl binding position of cyclodextrin residue to triazine has not been determined) on the fused silica. The stripping of the SAM from the surface in the presence of N-methylaminoethylpyridine is consistent with the strong basicity and nucleophilicity of this reactant. Attack of the N-methylaminoethylpyridine directly on the Si site of the siloxane SAM, if it occurs, would cleave the grafted 2-aminopropyl-4,6-dichloro-S-triazine organofunctional group from the surface. Alternatively, formation of hydroxide ion at the fused silica surface, which can also attack the Si site, via deprotonation of residual adsorbed water in the SAM by the basic N-methylaminoethylpyridine reactant would also lead to cleavage of the organofunctional group. Because the hydroxyl groups of the β-cyclodextrin reactant are insufficiently basic or nucleophilic to attack the Si site of the SAM, formation of surface-bound 2-aminopropyl-4-β-cyclodextrin-6-dichloro-S-triazine material, rather than cleavage of the of the 2-aminopropyl-4,6-dichloro-S-triazine organofunctional group of the SAM occurs.

Regardless of the mechanism for stripping the SAM from the surface, it is clear that the choice of reactants and reaction conditions are critically important for successful grafting of materials potentially useful as self-cleaning or self-decontaminating films to silica, alumina, and related oxide surfaces and processes for doing so are not often straightforward. Consequently, there exists a clear need to develop such means for the protection of metal surfaces. It is our intention in this disclosure to describe various self-cleaning or self-decontaminating coatings capable of providing protection against chemical and biological threats through catalytic degradation of chemical or microbial contaminants in contact with said coatings on metals as well as means for applying said coatings to metal surfaces that surmount the problems associated with the presence of metal oxide films on said metals.

### BRIEF DESCRIPTION OF THE FIGURES

FIG. 1: Polymer Net Surface Microbial Protection Coating Bearing n-Alkyl Quaternary Ammonium Salt Groups. The purported cell wall penetration lysing mechanism is shown. The blue- and red-striped layers and light blue ovals represent the protected surface, comprising in this case oppositely-charged polyelectrolyte layers (striped layers) coating a roughened substrate designated by the underlying light blue ovals.

FIG. 2: ACHT Structure.

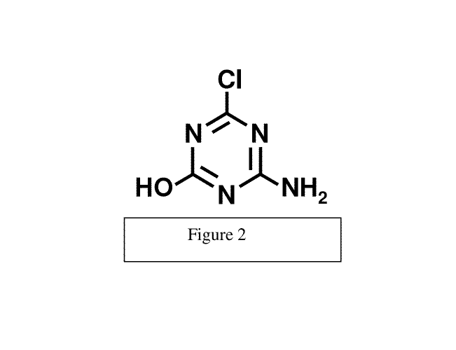

FIG. 3: Structures of Cyanuric Chloride (Left) and Hexachlorocyclotriphosphazene (Right).

FIG. 4: Absorption Spectrum of Surface-bound 2-aminopropyl-4,6-dichloro-S-triazine Material on Fused Silica.

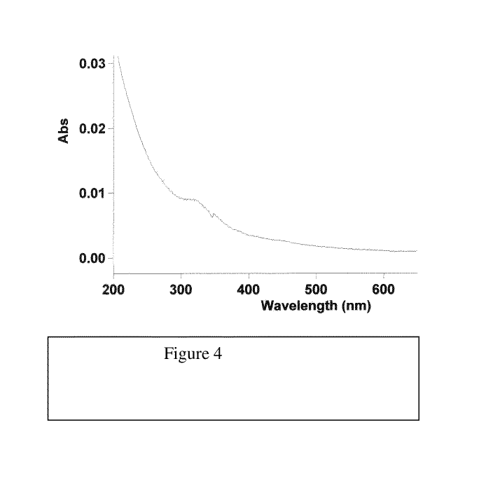

FIG. 5: Method for Fabrication of Multilayer Films From Oppositely-charged Polyelectrolytes via Layer-by-Layer Electrostatic Assembly.

FIG. 6: Structures of Some Representative Polyelectrolytes Useful for Fabrication of Self-cleaning or Self-Decontaminating Films Comprising Polyelectrolyte Multilayers.

FIG. 7: Fabrication Scheme for Self-cleaning OPH-Multilayer Films for Protection of Aluminum Substrates by Catalytic Degradation of Pesticide Contaminants. OPH is organophosphorous hydrolase enzyme and BTP is pH ˜8.6 bis-trispropane buffer. The subscripts “w” and “b” indicate aqueous solution and solution containing BTP buffer, respectively.

FIG. 8: Reaction Scheme for Attachment of Chloromelamine Residue and n-Alkyl Quaternary Ammonium Salt to a Single Mixed Polyelectrolyte.

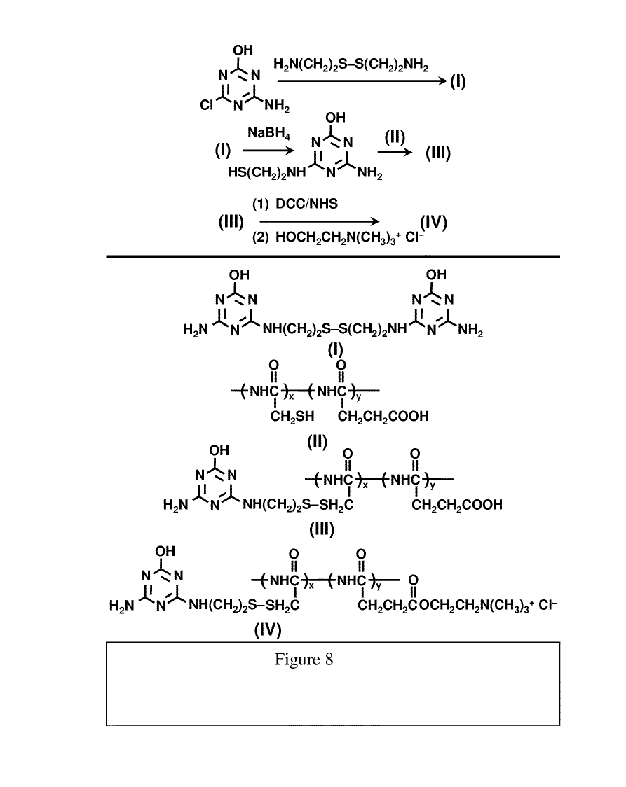

FIG. 9: Alternative Reaction Scheme for Attachment of Chloromelamine Residue and n-Alkyl Quaternary Ammonium Salt to a Single Mixed Polyelectrolyte.

FIG. 10: Reaction Scheme Using Triazine Residue as a Carrier for Both Passive and Active Microbial Degradation.

### DESCRIPTION

Self-cleaning or self-decontaminating films useful as coatings for metal surfaces must possess the ability to degrade chemical and/or microbial contaminants in contact with said films. Said contaminants are preferably degraded in a catalytic manner. By the term “catalytic”, we mean that the films or a component or components thereof are capable of eliminating contaminant species upon contact with said film repeatedly, without the need for additional reagents or intervention by personnel to maintain the abilities of said films to degrade contaminants.

Films capable of degrading contaminants in a non-catalytic manner are also useful. By the term “non-catalytic”, we mean that although the film or a component or components thereof become inactive after a single cycle of decontamination of a contaminant in contact with said film, the self-cleaning or self-decontaminating activity of said film can be easily regenerated by contact of an activating reagent with the film. For example, chloramine-based antimicrobial films described in further detail below are converted to unreactive melamines in the process of killing microbial life forms attached to said films.

However, the chloramines functional group can be easily and repeatedly regenerated in the film by rinsing with a bleach solution. Consequently, such films are effective in providing protection against bacterial contamination for metal surfaces in public areas or food preparation areas, where regular cleaning protocols are required using dilute bleach solutions. Both “catalytic” and “non-catalytic” films are described in further detail below.

Self-cleaning or self-decontaminating films for the protection of metal surfaces from chemical or biological hazards, whether catalytic or non-catalytic, share several requirements and features. In all such cases, the films must first adhere well to the substrate to prevent delamination and loss of protection for the metal surface. Likewise, said films must possess sufficient abrasion resistance during normal use of the coated metal surface to maintain protection throughout the lifetime of the application. The films should also ideally be colorless and transparent. This feature is desirable for cosmetic and security reasons. For example, in the manufacture of modern appliances, the anodized aluminum exterior of the appliance imparts a distinctive color and/or texture to the surface desirable to the consumer; therefore, the film should not affect the appearance. This requirement may preclude the use of multilayered films containing Ag(0) colloids as effective antimicrobial films in this application because the Ag(0) colloids impart a color to the surface by virtue of their plasmon resonance absorption bands in the visible spectral region.

Likewise, a transparent film provides obvious security advantages in connection with the protection of aluminum handrails, elevator panels, etc. . . . in public areas from contamination by deliberate release of chemical or microbial contaminants. Specifically, the uncertainty as to whether an area is or is not protected by such a film renders the selection of a target by a terrorist or other individual bent on causing harm to the public more difficult, since the objective of said persons is to create a maximal amount of panic and damage.

The aforementioned properties can be achieved using polyelectrolyte multilayer films comprising layered polyelectrolytes having the proper chemical functional groups as portions of their chemical structure to simultaneously promote adhesion, maintain transparency, and build abrasion resistance via interlayer crosslinking, while also providing directly the ability to neutralize chemical and/or biological threats or encapsulate materials that can do so. Now described are composite multilayer films that offer these capabilities by virtue of their component polyelectrolyte layers and combinations and arrangements thereof.

For films capable of chemical or biological threat protection, a key to fabricating effective films is to ameliorate the deleterious effects associated with the presence of the metal oxide, via separation of the active film components responsible for neutralizing the chemical or biological threats from the oxide surface. This can be done by fabricating a buffer layer comprised of multiple polyelectrolyte layers between the metal oxide surface and the active elements of the film. The fabrication method most often used exploits the natural electrostatic attraction of charged polyelectrolytes to oppositely charged surfaces to fabricate multilayered films via a layer-by-layer approach (G. Decher, Science 1997, 277, 1232).

Multilayer fabrication requires dipping a charged substrate into a solution containing an oppositely charged polyelectrolyte. Electrostatic attraction binds charged regions of the polyelectrolyte to the opposite surface charges. As a result, adsorption of a monolayer thin film of polyelectrolyte occurs. However, because of the steric constraints of the polymer backbone, all charges on the polyelectrolyte cannot pair with surface charges. Consequently, the net charge on the polyelectrolyte-covered surface is reversed due to the presence of these uncompensated polyelectrolyte charge sites. Through alternating treatments of the substrate with solutions containing oppositely-charged polyelectrolytes, a structured multilayer film is eventually deposited.

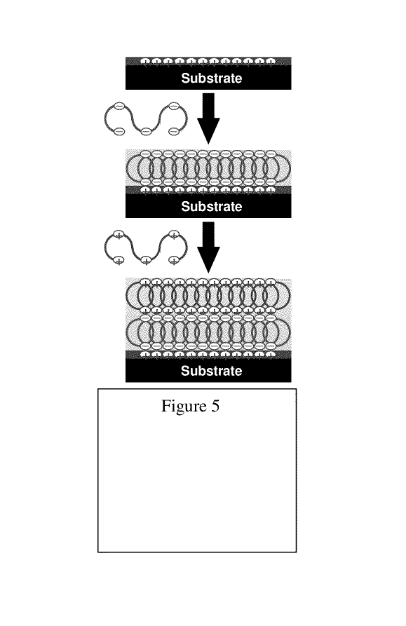

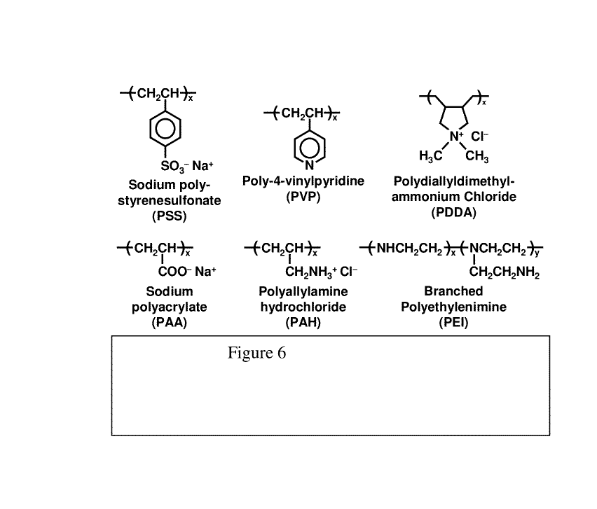

As an example, FIG. 5 illustrates multilayer fabrication on a positively-charged substrate. The initial positive charge on the substrate surface is generated via control of the solution pH, as in the case of silica, alumina, and related oxides having distinct isoelectric points, or chemisorption of naturally charged materials as self-assembled monolayers (SAMs). Adsorption of negatively-charged polyelectrolytes in this case (i.e., red strands), such as polyacrylate (PAA) or polystyrene sulfonate (PSS), to a positively-charged substrate forms a polyelectrolyte layer having a net negative surface charge (i.e., pink layer). If the substrate is now dipped into a solution containing a positively-charged polyelectrolyte (i.e., blue strand) like polyethylenimine (PEI), polyallylamine (PAH), or polydiallyldimethylammonium chloride (PDDA), a new polyelectrolyte layer electrostatically adsorbs and reverses the net surface charge again, restoring the original positive surface charge of the substrate. Dipcoating (P. T. Hammond, Curr. Opin. Colloid Interface Sci. 1999, 4, 430), spraycoating (J. B. Schlenoff, et. al., Langmuir 2000, 16, 9968), and spincoating (P. A. Chiarelli, et. al., Langmuir 2002, 18, 168) methods have been employed to treat substrates to fabricate multilayers in this manner and are applicable for use. Structures of some polyelectrolytes having good visible transparency useful for the practice are shown in FIG. 6. Note that the structures in FIG. 6 are not meant to limit the scope and are representative, rather than all-inclusive, structures useful for practicing.

Because the initial few layers of polyelectrolyte deposited according to the method of FIG. 5 usually do not completely cover the oxide surface due to surface roughness and inhomogeneous distributions of oxide surface chemical functional groups, multiple layers of polyelectrolyte are deposited. In practice, usually ≧˜6 polyelectrolyte layers (i.e., 3 polycationic and 3 polyanionic layers alternately deposited per FIG. 5) are deposited as a buffer to sufficiently separate the metal oxide layer form the components of the film, such as enzymes or chemical species as described below, active towards the degradation of chemical and biological threats.

Adhesion of these initial polyelectrolyte layers to the metal oxide can be important. The polyelectrolytes are chosen such that strong binding via electrostatic, hydrogen bonding, and/or van der Waals interactions can occur between the oxide substrate and the first polyelectrolyte layer(s), as well as between polyelectrolytes in adjacent film layers. Initial adsorption of the first polyelectrolyte layer directly to the substrate oxide can be done if desired. In this case, the polyelectrolyte is chosen and the pH of the polyelectrolyte solution is ideally adjusted such that it is greater than or less than the oxide pI to create a charged oxide surface opposite in charge to the polyelectrolyte. For example, for a deposition pH<pI, the net positive surface potential (i.e., charge) of the oxide best requires the use of an anionic polyelectrolyte to maximize polyelectrolyte adsorption to the oxide surface via attractive electrostatic binding interactions and vice versa.

In general, direct binding of polyelectrolyte to the oxide layer provides acceptable adhesion because each polyelectrolyte chain is electrostatically bound to the oxide surface by multiple strong electrostatic interactions. However, improvements in adhesion of the polyelectrolyte films can often be accomplished if desired by using SAMs. Appropriate SAMs are formed via chemisorption to the oxide surface of a hetero- or homo-bifunctional moiety comprising a reactive group joined to a charged group through an inert linker species. The reactive group is chosen to chemisorb readily to the oxide surface and may include trihalosilane, trialkoxysilanes, carboxylic acids, and phosphonic acids, with phosphonic acids most preferred for alumina. The charged group, including but not limited to protonated alkylamines, tetraalkylammonium salts, tetraalkylphosphonium salts, pyridinium salts, organocarboxylates, organosulfonates, and organosulfates, provides a charged site for adsorption of an oppositely-charged polyelectrolyte layer. Note that charged species capable of chemisorbing to the oxide layer, such as carboxylates or phosphonates, can also function as the charged group for interaction with the polyelectrolyte. The linker group is typically a chemically inert n-alkyl chain containing 2 or more carbon atoms or an aromatic phenyl group (typically 1, 4-disubstituted) or combination thereof. The use of SAMs provides at least two advantages in the fabrication of the multilayer film: (1) SAMs effectively increase the surface density of charged groups available for interaction with the polyelectrolyte, particularly in the case of SAMs prepared using trialkoxy- or trihalosilane chemisorption agents, and; (2) SAM chemisorption provides a covalently-bound layer on the oxide having a fixed or pH-controllable charge determined by the nature of the charged group present.

The adhesion of the buffer polyelectrolyte multilayer to the oxide can be further improved via crosslinking of the component polyelectrolyte layers, either during the deposition of each layer or after the buffer layer has been fabricated. For example, for multilayers formed via the alternate deposition of polyallylamine hydrochloride (PAH) and polyacrylate (PAA), cross-linking is readily accomplished by conversion of a portion of the carboxylic acid groups of the polyacrylate to N-hydroxysuccinimide esters prior to use of the polyelectrolyte to fabricate the multilayer, as is well known to organic chemists. During or after multilayer fabrication, reaction of the active ester with a portion of the primary amines from the adjacent polyallylamine layers leads to crosslinking via covalent amide bond formation. A similar result can be accomplished by infusing a pH-adjusted water-soluble carbodiimide (CDI)/water-soluble N-hydroxysuccinimide (NHS) solution into a completed polyallylamine-polyacrylate multilayer film after fabrication containing a portion of free carboxylic acid groups unbound by amines (such films can be prepared by using a PAA solution having ˜2.5<pH<˜4.5), as described herein (T. C. Wang, et. al., Langmuir 2002, 18, 3370-3375).

Simple heating of the polyallylamine-polyacrylate multilayer can also lead to partial crosslinking and film stabilization (see, e.g.; J. J. Harris, et. al., J. Am. Chem. Soc. 1999, 121, 1978). As an alternative, crosslinking of alkylamines using a diisocyanate crosslinker within a multilayer assembly has also been reported (E. R. Welsh, et. al., Langmuir 2004, 20, 1807) and is a viable option for our application, together with the use of other known amine crosslinking agents like glutaraldehyde, since interpenetration of polyelectrolyte layers within the multilayers occurs rendering amine bridging in adjacent layers of amine-functionalized polyelectrolytes within the multilayer possible.

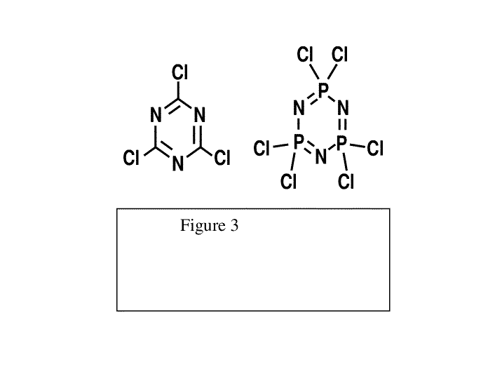

The use of crosslinking agents of controlled reactivity, specifically cyanuric acid chloride or hexachlorocyclotriphosphazene derivatives (note FIG. 3), provides yet another means to crosslink polyelectrolyte layers within multilayer films. For example, the Cl atoms of cyanuric acid chloride are sequentially displaced by nucleophiles, such as primary amines, at increasingly higher temperatures (e.g., the first Cl is displaced at room temperature, the second that ˜60-80° C. and the third at ≧˜100° C.). Consequently, a small fraction (e.g., <˜20%) of the primary amines present in the PAH polyelectrolyte can each be reacted with the first Cl of cyanuric acid chloride species to generate a 2-PAH-4,6-dichloro-S-triazine derivative.

Because a majority of the primary amines remain unreacted, the resulting species remains sufficiently protonated and soluble in water (pH<˜8) for use in fabricating multilayer films via the electrostatic layer-by-layer method of FIG. 5. Once such a multilayer is formed, heating reacts the second Cl at ˜60-80° C. and, if desired, the third Cl at ≧˜100° C. with available amine groups from nearby polyelectrolyte layers to efficiently crosslink the multilayer. The six Cl groups of hexachlorocyclotriphosphazine cannot all be sequentially reacted as is the case for cyanuric acid chloride. Typically, the first (i.e., primary) Cl group on a particular P site reacts more quickly and under milder conditions than the second (i.e., secondary) Cl group. In addition, although the primary Cl groups generally react collectively prior to the secondary Cl groups, the degree of Cl substitution can be sufficiently controlled to permit polyelectrolyte crosslinking through judicious choice of the reaction stoichiometry and conditions (e.g., temperature and solvent) (I. Dez, et. al., Macromolecules 1997, 30, 8262; E. T. McBee, et. al., Inorg. Chem. 1966, 5, 450; K. Ramachandran, et. al., Inorg. Chem. 1983, 22, 1445).

Normally, electrostatic interactions between oppositely-charged polyelectrolyte layers are used to bind the multilayer together. However, other interactions such as hydrogen bonding may also be used (E. Kharlampieva, et. al., Macromolecules 2003, 36, 9950). For hydrogen-bonded multilayer systems, such as those formed by interactions between acrylic acid and acrylamide functionalized species, thermal crosslinking leading to imidization to stabilize the resulting films is also possible (S. S. Yang, et. al., J. Am. Chem. Soc. 2002, 124, 2100). Photochemical cross-linking reactions can also be used to conveniently crosslink the film under mild conditions, especially in cases where the use of crosslinking agents such as CDI might chemically degrade the film or thermal reactions might damage the structure of the film. For example, polycationic diazo resins are well known to covalently crosslink with polyacrylate films during UV light exposure (J. Sun, et. al., Langmuir 2000, 16, 4620).

Having described a suitable polyelectrolyte multilayer buffer to ameliorate the potentially deleterious effects due to interactions of the metal oxide of the substrate with the active elements, such as enzymes or reactive chemical functional groups, required to provide the self-cleaning or self-decontaminating functions of the film, now described are these self-cleaning and self-decontaminating functions. Specifically, additional layers having the abilities to provide the self-cleaning or self-decontamination functions are fabricated directly on the multilayer buffer film via adaptations of the process shown in FIG. 5. For example, a self-cleaning or self-decontaminating film capable of catalytically hydrolyzing organophosphorous pesticides is readily fabricated on an aluminum surface bearing a multilayer buffer film via alternatively dipcoating of PEI and organophosphorous hydrolase (OPH) enzymes at pH ˜8.6, where PEI remains a polycation and OPH is negatively-charged and sufficiently stable in aqueous solution for deposition. However, fabrication in this manner leads to variable levels of enzyme deposition and, therefore, films of variable composition and activity.

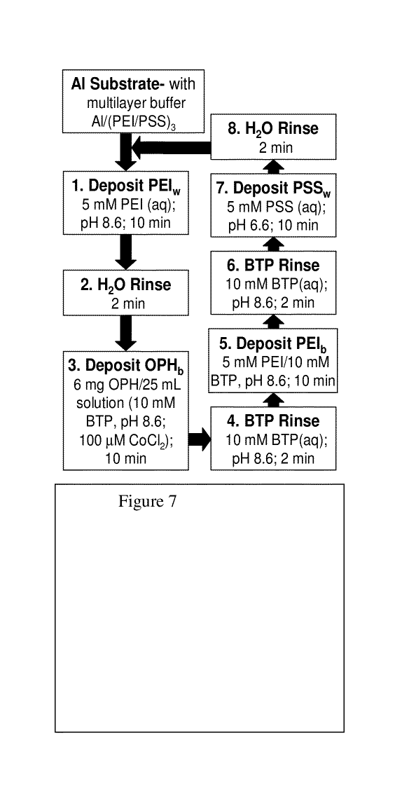

While films catalytically active towards degradation of pesticides, such as MPT, can be prepared in this manner, FIG. 7 illustrates a more reproducible means and preferred for fabricating such films. In FIG. 7, PEI and PSS polyelectrolyte layers, together with OPH, are employed as film components. Note that in FIG. 7, the deposition of additional OPH enzyme layers can be done by interspersing a PSS layer as a negatively-charged separation layer between the adjacent OPH layers. In this manner, the OPH is more reproducibly deposited (˜±15%), leading to fabrication of films having more reproducible and predictable pesticide hydrolysis kinetics and characteristics.

Films fabricated using the scheme shown in FIG. 7 are evaluated their effectiveness in the catalytic degradation of MPT pesticide in a test solution comprising 100 μM MPT is 80:20 v/v methanol/10 mM CHES pH 8.6 buffer (aq) (where CHES is 2-[N-cyclohexylamino]ethane sulfonic acid). Specifically, untreated Al pieces as the silver-colored plates in front of the central test tube and the OPH multilayer-coated Al samples as the gold-colored pieces in front of the right-most test tube. The OPH multilayer-coated Al samples have the film structure: Al/(PEI/PSS)₃/(PEI/OPH/PEI/PSS)₃. The gold color of the samples is the result of using partially purified OPH enzyme, which contains yellow protein residue that co-deposits with the OPH during film fabrication, to prepare the samples. This mode of preparation was deliberately selected to provide a visual confirmation of the enzyme deposition during film fabrication. Subsequent experiments using purified OPH enzyme provide colorless, yet catalytically active, films (not shown), as required for many of the applications discussed herein.

The activity of the films during a 7 day test at room temperature the MPT solution in the test tubes. The leftmost test tube contained only MPT control solution, which did not contact the untreated or multilayer-coated Al samples, and remains colorless. Likewise, the central test tube solution, which was in contact with the untreated Al samples, also remains colorless. In contrast, the rightmost test tube MPT solution, which contacted the OPH multilayer-coated Al sample, is pale yellow in color. Spectrophotometric analysis of the solution indicates that the yellow color (λₘₐₓ=399 nm) is due to p-nitrophenol (PNP), generate by the catalytic hydrolysis of MPT by the film. Repetition of the experiment using fresh MPT solution indicates that the OPH multilayer-coated Al samples retain their catalytic activity for at least 3 cycles of use.

Noted here are several additional points regarding these types of films. First, the method is obviously not restricted to organophosphorous hydrolase as the enzyme, nor PSS and PEI as the polyelectrolyte components. Other enzymes capable of hydrolyzing pesticides and nerve agents may certainly be incorporated, particularly enzymes, derived from thermophile life forms, exhibiting improved catalytic activities at high temperatures. Such enzymes may also include genetically engineered variants of OPH and its cogeners designed to retain catalytic activities under the presence of extreme environments (e.g., high salt levels or organic solvents). Enzymes capable of neutralizing other hazards will also be useful, e.g., the encapsulation of mustardase enzymes isolated from Caldariomyces fumago fungus (Professor M. Tien, Department of Biochemistry, Penn State University, University Park, Pa., personal communication) or Rhodococcus bacteria (S. P. Harvey, “Enzymatic Degradation of HD”, Program Final Report ERDEC-TR-2001, Edgewood Research and Development Engineering Center, U.S. Army Armament Munitions Chemical Command, Aberdeen Proving Ground, MD 21010-5423) for the hydrolysis of mustard gas and related contaminants.

In addition, since genetic variants of OPH isolated from different species hydrolyze structurally dissimilar pesticides at different rates, a cocktail of enzymes is most useful to provide broad spectrum protection against surface contamination by organophosphorous pesticide residues of unknown composition and source. Of course, the enzyme cocktail may be encapsulated as a mixed enzyme layer within a multilayer film or each different enzyme may be present as a separate layer.

Second, the methods described above leading to improvements in film adhesion and abrasion resistance may also be applied to the enzyme-multilayer portions of the protective film composite, provided that care is taken to choose methods that do not materially damage the ability of the enzyme to function. For example, although thermal crosslinking typically denatures enzymes, certain chemical crosslinking methods are compatible. In particular, the structure of OPH enzyme indicates that there are no cysteine groups present near the enzyme active site (S. Gopal, et. al., Biochem. Biophys. Res. Commun. 2000, 279, 516). Consequently, alkylthiol derivatives can be used as crosslinking agents during or after assembly of the multilayer film to provide crosslinking via formation of covalent disulfide bonds between adjacent thiol sites without undue fear of destroying the active site of the OPH.

For example, a fraction (typically <˜20%) of the primary (and secondary) amine residues of PEI are reacted with a water soluble N-hydroxysuccinimide ester of thioacetic acid to graft alkylthiol groups to the PAH polymer chain via amide bind formation. Likewise, a similar amide formation reaction is carried out using 2-aminoethanthiol and the sulfonyl acid chloride of PSS. Because the degree of substitution in each case is low, each polyelectrolyte retains sufficient charge and water solubility to fabricate multilayer films. However, the presence of alkylthiol side chains is sufficient to induce cross-linking between adjacent polyelectrolyte layers within the multilayer via disulfide bond formation, increasing the degree of adhesion to the substrate (i.e., multilayer buffer coating in this case) and durability.

Additional improvements accrue through use of OPH enzymes genetically engineered to possess cysteine residues capable of forming similar disulfide bridges with alkylthiol side chains of adjacent polyelectrolyte on the specific locations (not interfering with the active site) on the OPH surface. Improved film integrity and durability, as well as enzyme resistance to denaturation by high salt solutions and organic solvents, can also accrue via capping of the film with a crosslinked, semi-permeable polymer net. For example, electrostatic adsorption of N-2-aminoethyl-3-aminopropyltrimethoxysilane onto a PAA terminated multilayer film readily occurs in aqueous solution near pH ˜7. Through a subsequent increase in solution pH, hydrolysis of the trimethoxysilane groups to trisilanol groups occur, followed by formation of covalent siloxane bonds crosslinking the surface. OPH enzymes present in multilayer films capped in this manner retain activity towards pesticide hydrolysis, albeit at diminished levels, even after 2 hr pre-treatments with 2 M NaCl (aq) solutions or pure acetone solvent (Y. Lee, et. al., Langmuir 2003, 19, 1330).

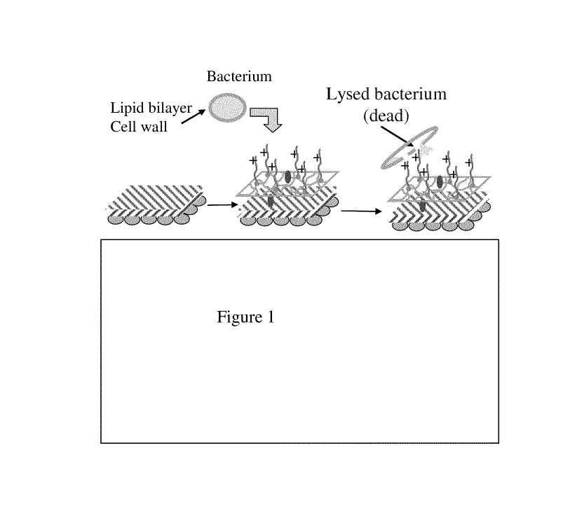

Self-cleaning and self-decontaminating multilayer films for the protection of metal surfaces from microbial contamination having acceptable adhesion, durability (abrasion resistance), and transparency can be similarly fabricated. In this case, both catalytic and non-catalytic protection modes are readily accommodated. Catalytic systems are most readily formed by using a water soluble cationic polyelectrolyte containing pyridinium, quaternary ammonium, or quaternary phosphonium salt functional groups as a portion of its structure as the outermost layer of the multilayer film (i.e., the layer last deposited). The n-alkyl chain associated with these materials is typically 2-20 carbon atoms in length, more preferably ˜4-18 carbon atoms in length, and even more preferably ˜12-16 carbon atoms in length, such that death of a microbe contacting the surface is facilitated via penetration of the alkyl chain into the bilayer comprising the cell wall, resulting in lysing of said cell wall and subsequent cell death as illustrated in FIG. 1 (S. B. Lee, et. al., Biomacromolecules 2004, 5, 877).

For N-containing polyelectrolyte layers bearing alkylpyridinium groups, a surface density of ≧˜10¹² alkylpyridinium N⁺/cm² is preferred and a surface density of ≧˜10¹⁴ alkylpyridinium N⁺/cm² is most preferred to ensure immediate microbe death on contact with the surface (R. Kügler, et. al., Microbiology 2005, 151, 1341; L. Cen, et. al., Langmuir 2003, 19, 10295). Alternatively, the requisite n-alkyl pyridinium, quaternary ammonium, or quaternary phosphonium salts may be formed via reaction of the outermost polyelectrolyte layer of a multilayer film with an appropriate alkylating agent or reactant to form the desired salt on the multilayer surface using techniques well-known to organic chemists.

For example, treatment of a multilayer comprising an outermost PAH or PEI layer with a water soluble N-hydroxysuccinimide ester of a halide salt of ω-trimethylammonium hexanoic acid leads to formation of an amide bond and covalent grafting of a linear six-carbon alkyl chain terminated by the trimethylammonium group salt to the PAH or PEI layer Likewise, alkylation of a pyridine group of a multilayer comprising an outermost polyvinylpyridine layer occurs following reaction with n-butyl iodide in DMF, provided that the underlying multilayer has been sufficiently covalently crosslinked using methods similar to those described herein to stabilize it against dissolution and delamination from the metal surface during the reaction.

Non-catalytic systems can also be prepared and two representative examples capable of regeneration of catalytic activity after use for substrate re-use are given here. First, melamines similar in structure to ACHT can be incorporated into or onto the surfaces of the multilayer films using modified literature protocols (Y. Sun, et. al., Ind. Eng. Chem. Res. 2005, 44, 7916; M. Braun, et. al., J. Polym. Sci. A—Polym. Chem. 2004, 42, 3818) to provide antimicrobial protection to the underlying metal substrate. A variety of chemical approaches known to organic chemists are available for this purpose, dictated primarily by the chemical nature of the polyelectrolyte and the melamine derivative. Once again, as mentioned herein, the role of the substrate (in this case the outermost polyelectrolyte layer of the multilayer) can adversely affect the course of a reaction. For example, stepwise fabrication of the desired melamine structure by sequential reaction involving the initial grafting of cyanuric chloride to an alkylamine in the outermost PAH or PEI polyelectrolyte of a multilayer film is prohibitively difficult. While the first Cl of the cyanuric acid chloride readily reacts, attempts to substitute the second Cl are froth with complications. Specifically, the high effective local concentration of additional amine present on the polyelectrolyte surface can effectively compete with solution reagent (such as ammonia of hydroxide) for displacement of the Cl, leading to product mixtures that can effectively alter the efficacy of the resulting material as an antimicrobial agent.

While reaction conditions can sometimes be adjusted to compensate for this problem, a more preferable approach builds much of the desired melamine structure prior to attachment to the polyelectrolyte. Because displacement of successive Cl atoms in cyanuric acid chloride occurs requires increasingly harsh reaction conditions, an approach can be to replace the first Cl with a desired substituent, such as NH₂, by room temperature reaction. If it is desired to maintain one Cl site on the final product, the material can be directly reacted at somewhat higher temperatures (e.g., ˜60-80° C.) with the amine site of the polyelectrolyte, either as a portion of the existing multilayer film or in solution. In this example the 4-amino-6-chloro-S-triazine residue is grafted at the 2-position to the amino group of the PAH (or PEI).

If the reaction is run under solution conditions, this 2-PAH (2-PEI)-4-amino-6-chloro-S-triazine product is available for use in building the multilayer film, provided that sufficient unreacted PAH (or PEI) alkylamine sites remain available for electrostatic attraction (in their protonated form) to the anionic polyelectrolyte component and to ensure water solubility required for the dipcoating process. The presence of such a material as an internal polyelectrolyte component of the multilayer is advantageous because the unreacted Cl becomes reactive at higher temperatures (e.g., >˜100° C.), permitting the multilayer to be internally crosslinked via Cl displacement by amine or hydroxyl groups in adjacent polyelectrolyte layers. Of course, both the first and second Cl sites of cyanuric chloride can be sequentially reacted prior to attachment of the resulting species to the polyelectrolyte via displacement of the third Cl, if desired. For example, sequential reaction of cyanuric chloride with ammonia, hydroxyl ion, and a cellulose hydroxyl group leading to 2-O-cellulose-4-amino-6-hydroxy-S-triazine provides a known method of grafting an antimicrobial melamine precursor to cotton fabric (M. Braun, et. al., J. Polym. Sci. A—Polym. Chem. 2004, 42, 3818). Use of an appropriately charged polysaccharide derivative, such as heparin sulfate or chitosan, can provide a modified polyelectrolyte suitable for multilayer fabrication.

The treatment of suitably stabilized (e.g., crosslinked) multilayers bearing melamine groups of structure similar to ACHT with an aqueous bleach solution effectively chlorinates the melamine NH₂ group, forming a chloromelamine species that activates such films as antimicrobial agents. Release of Cl in the presence of a microbe effective kills said microbe, maintaining the protection of the underlying metal surface against microbial contamination to the extent that active chloromelamine residues remain on the multilayer. The incorporation of chloromelamine residues on polyelectrolyte layers within the multilayer offers additional layers of protection under the proper conditions. Microbes are known to exert influence on the structure of a surface as they attach to said surface and begin to colonize it. For example, colonization of microbial life forms on the hulls of seafaring vessels is known to encourage hull corrosion. As microbes contaminating a multilayer-protected surface modify the morphology of the multilayer film, additional chloromelamine residues originally buried within interior polyelectrolyte layers will ultimately contact the microbes and kill them, provided that the degree of crosslinking is sufficiently low (e.g., preferably >˜2% and <˜20%, depending on the properties of the polyelectrolytes as is known to person skilled in the art of polymer applications) to permit limited conformational lability of the multilayer without adversely affecting multilayer adhesion or durability).

In either case, because the incorporation and loss of Cl at the melamine NH₂ group is reversible, treatment of surfaces modified by such ACHT derivatives with an aqueous bleach solution can regenerate the active chloromelamine agent and protection for the underlying metal surface. Consequently, for aluminum metal surfaces in public areas or food service areas, where regular cleaning regimens are mandated by law, said multilayer films bearing ACHT-derivative structures can provide extended protection against microbial contamination between cleaning cycles.

An additional non-catalytic surface offering protection against microbial contamination comprises a Ca²⁺ and/or Mg²⁺ ion-ligating functional group, including but not limited to humates (J. G. Hering, et. al., Environ. Sci. Technol. 1988, 22, 1234-1237), phosphatidylcholines (K. K. Yabusaki, Biochemistry 1975, 14, 162), and β-hydroxyquinoline derivatives (G. Persaud, et. al., Anal. Chem. 1992, 64, 89) as a component of said surface. The presence of such ligands at the multilayer surface offers the possibility of lysing the microbial cell wall by competitive binding and extraction of the Ca²⁺ and/or Mg²⁺ ions in the microbial cell wall that function as the “glue” maintaining the cell wall integrity (R. Kügler, et. al., Microbiology 2005, 151, 1341), provided that such ligands are able to sufficiently penetrate said cell walls.

Once again, attachment of said ligands to the outermost polyelectrolyte layer of the multilayer is required, either by direct grafting of said ligand to said outermost polyelectrolyte layer or by chemical modification of the desired polyelectrolyte, followed by use of said modified polyelectrolyte to complete the fabrication of the multilayer film. An n-alkyl chain typically of ˜2-20 carbon atoms in length connecting the ligand group to the polyelectrolyte permits sufficient penetration of the alkyl chain into the bilayer comprising the cell wall to allow the ligand access to the Ca²⁺ and/or Mg²⁺ ions in the microbial cell wall, as required for complexation.

Upon disruption of the microbial cell wall and cell death by complexation of the Ca²⁺ and/or Mg²⁺ ions in the microbial cell wall by the multilayer surface ligand, the multilayer ligand must be regenerated. This can be done via use of a cleaning solution, as similarly described above for the regeneration of chloromelamine derivative. In this case, however, bleach is not used to regenerate the ligand binding capacity. Instead, multilayer surface is treated with a ligand, such as ethylenediaminetetraacetic acid (EDTA), which complexes the Ca²⁺ and/or Mg²⁺ ions much more strongly in basic solution than the multilayer surface ligands. As a result, the Ca²⁺ and/or Mg²⁺ ions are extracted from the multilayer surface ligand by the EDTA in the rinse/wash solution, regenerating the multilayer surface ligand's ability to again bind and extract Ca²⁺ and/or Mg²⁺ ions from the microbial cell wall. An aqueous solution having pH >˜8 and an effective concentration of ˜0.1-1.0% wt. EDTA can successfully extract Ca²⁺ and/or Mg²⁺ ions complexed by multilayer surface ligands appropriate for use.

A preferred means to produce multilayer films having antimicrobial properties according to the methods involves the grafting of both passive and active antimicrobial agents to the multilayer film. This can be accomplished through two primary means. The first involves separately binding an appropriate n-alkylpyridinium salt, quaternary ammonium salt, or quaternary phosphonium salt, or combinations thereof, to one type of functional group on a polyelectrolyte bearing two reactable functional groups of orthogonal reactivity (i.e., reactions that can be performed at the first functional group will leave the second functional group unchanged, and vice versa) either prior to or after the polyelectrolyte is deposited as the outermost polyelectrolyte layer in the multilayer film. Following binding of the passive antimicrobial component, the second functional group of the polyelectrolyte is separately reacted to covalently bind an active component, such as a melamine derivative or a ligand capable of binding Ca²⁺ and/or Mg²⁺ ions. Of course, the active component can be bound to the polyelectrolyte prior to binding the passive component, provided that reaction conditions amenable to the sequence can be found, such as are well-known to synthetic organic chemists (e.g., the product of the first reaction must be soluble and non-reactive in a solvent suitable for grafting the second component).

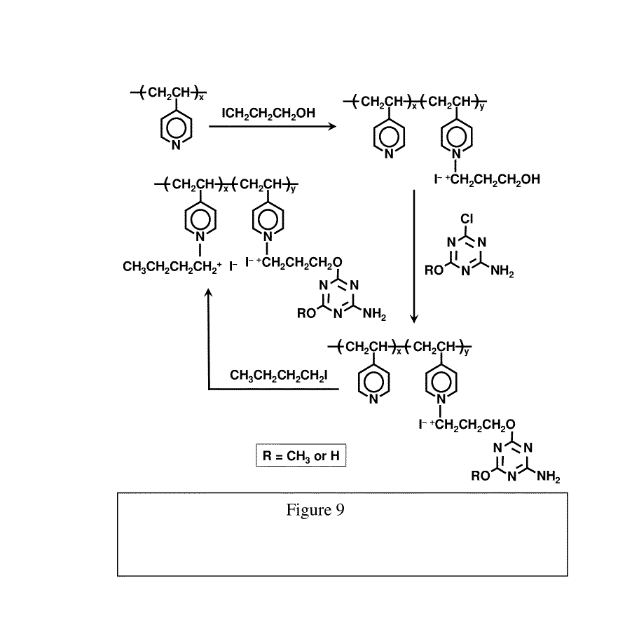

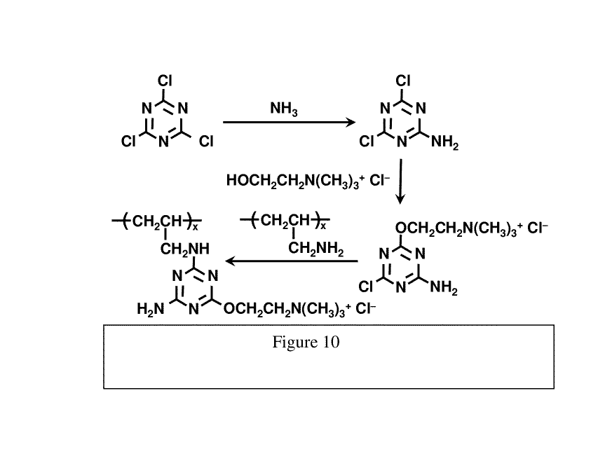

In addition, the chemical sequence selected must yield either a cationic or anionic water soluble polyelectrolyte to permit electrostatic layer-by-layer multilayer film fabrication using the final reaction product. Finally, the surface density of passive functional groups based on n-alkyl quaternary ammonium salt, pyridinium salt, or quaternary phosphonium salt preferably should remain sufficiently high (e.g., preferably ≧˜10¹⁴ alkylpyridinium N⁺/cm² for alkylpyridinium species) such that rapid lysis and cell death is obtained on contact of a microbe with the multilayer film surface. For example, FIG. 9 shows a one such scheme for attachment of an ACHT derivative and N-alkyl quaternary ammonium salt to poly-cysteine-co-glutamic acid. Alternatively, a homogeneous polyelectrolyte can also be used, provided that similar conditions are satisfied. For example, FIG. 10 shows a scheme involving successive alkylations of pyridine N sites in PVP for attachment of both an ACHT derivative and formation of a quaternary butyl pyridinium salt.

Finally, perhaps one of the more efficient means to decorate the multilayer film surface with both passive and active microbial degradation functionalities is to utilize the triazine residue as a carrier for both. Specifically, FIG. 11 shows a reaction scheme in which a triazine residue prepared by the successive reaction of cyanuric acid chloride with ammonia and then choline produces a species containing both the passive n-alkyl quaternary ammonium species and active melamine amine group (for conversion to a chloromelamine with bleach). The attachment of this residue to PAH in FIG. 11 effectively packs both the passive and active microbial degradation functionalities onto a single primary amine side chain of the PAH.

The above description is that of a preferred embodiment of the invention. Various modifications and variations are possible in light of the above teachings. It is therefore to be understood that, within the scope of the appended claims, the invention may be practiced otherwise than as specifically described. Any reference to claim elements in the singular, e.g., using the articles “a,” “an,” “the,” or “said” is not construed as limiting the element to the singular.

1. A method of making a composite structure exhibiting the ability to degrade chemical or biological agents upon contact comprising:
coating a buffer film onto a substrate to be protected from the deleterious effects of chemical or biological agents possessing surface groups capable of deactivating materials having the ability to degrade chemical or biological agents, wherein said buffer film blocks the ability of said substrate surface groups to deactivate the materials having the ability to degrade chemical or biological agents and wherein said substrate is a metal substrate selected from the group consisting of aluminum, steel, and alloys thereof and having an oxide surface and wherein said buffer film consists of a multilayer chemically or physically bound to said substrate surface and wherein said buffer film comprises alternating layers of oppositely-charged cationic and anionic polyelectrolytes and wherein said cationic polyelectrolytes are selected from the group consisting of protonated polyethylenimine (PEI), polyallylamine hydrochloride (PAH), and polydiallyldimethylammonium chloride (PDDA) and wherein said anionic polyelectrolytes are selected from the group consisting of alkali metal salts of polyvinyl sulfate (PVS), polystyrenesulfonate (PSS), polyacrylate (PAA), and polymethacrylate (PMMA); and
coating a protective film onto said buffer film, wherein said protective film contains materials having the ability to degrade chemical or biological agents encapsulated in or comprising the outer surface of said protective film;
utilizing a triazine residue as a carrier for both passive and active microbial degradation functionalities;
decorating the multilayer film surface with both passive and active microbial degradation functionalities by utilizing the triazine residue; and
maintaining the appearance of the underlying substrate.

2. A method of making a composite structure exhibiting the ability to degrade chemical or biological agents upon contact comprising:
providing a substrate to be protected from the deleterious effects of chemical or biological agents possessing surface groups capable of deactivating materials having the ability to degrade chemical or biological agents;
coating a buffer film onto said substrate that blocks the ability of said substrate surface groups to deactivate the materials having the ability to degrade chemical or biological agents;
coating a first layer of protective film comprising positively charged polyelectrolytes onto said buffer film containing materials having the ability to degrade chemical or biological agents encapsulated in or comprising the outer surface of said protective film; and
coating a second layer of protective film comprising negatively charged polyelectrolytes onto said first layer of protective film wherein at least some of the constituent polyelectrolytes have been chemically modified to bear covalently attached materials capable of degrading biological agents as a portion of their structure;
wherein said substrate is a metal substrate having an oxide surface;
wherein said chemically modified constituent polyelectrolytes bear melamine derivatives wherein the melamine functional groups are prepared by reaction of aminoalkyl groups with reactive chlorine sites of one selected from the group consisting of 2-amino-4-chloro-6-hydroxy-S-triazine, 2-amino-4,6-dichloro-S-triazine, and 2,4-diamino-6-chloro-S-triazine;
wherein said melamine groups have reacted with said polyelectrolyte and wherein said protective film is terminated with a charged polyelectrolyte capping layer wherein said charged polyelectrolyte capping layer contains one selected from the group consisting of n-alkylpyridinium, n-alkylquaternary ammonium, n-alkylquaternary phosphonium functional groups, a ligand capable of binding Ca²⁺ and/or Mg²⁺ ions as a portion of its structure, and mixtures thereof;
wherein said chemically modified constituent polyelectrolytes bear melamine functional groups prepared by reaction of their available aminoalkyl groups with the reactive chlorine sites of one selected from the group consisting of 2-amino-4-chloro-6-hydroxy-S-triazine, 2-amino-4,6-dichloro-S-triazine, and 2,4-diamino-6-chloro-S-triazine;
wherein said melamine groups have been converted into chloromelamines by reaction of said amino sites of said melamine with bleach to render the protective film active for the degradation of biological agents; and
wherein said ligand is selected from the group consisting of humates, phosphatidylcholines, and β-hydroxyquinoline derivatives;
utilizing a triazine residue as a carrier for both passive and active microbial degradation functionalities;
decorating the multilayer film surface with both passive and active microbial degradation functionalities by utilizing the triazine residue; and
maintaining the appearance of the underlying substrate.

3. The method of making a composite structure exhibiting the ability to degrade chemical or biological agents upon contact of claim 2 further comprising the step of coating a third layer of protective film comprising positively charged polyelectrolytes onto said second layer of protective film and containing materials having the ability to degrade chemical or biological agents encapsulated in or comprising the outer surface of said protective film.

4. The method of making a composite structure exhibiting the ability to degrade chemical or biological agents upon contact of claim 3 further comprising the step of coating a fourth layer of protective film comprising positively charged polyelectrolytes onto said third layer of protective film and wherein at least some of the constituent polyelectrolytes have been chemically modified to bear covalently attached materials capable of degrading biological agents as a portion of their structure.

5. The method of claim 4 wherein said metal substrate is selected from the group consisting of aluminum, steel, and alloys thereof.

6. The method of claim 2 wherein said buffer film consists of a multilayer chemically or physically bound to said substrate surface and wherein said buffer film comprises alternating layers of oppositely-charged cationic and anionic polyelectrolytes.

7. The method of claim 4 wherein said positively charged polyelectrolytes of the buffer film are selected from the group consisting of protonated polyethylenimine (PEI), polyallylamine hydrochloride (PAH), and polydiallyldimethylammonium chloride (PDDA) and wherein said negatively charged polyelectrolytes of the buffer film are selected from the group consisting of alkali metal salts of polyvinyl sulfate (PVS), polystyrenesulfonate (PSS), polyacrylate (PAA), and polymethacrylate (PMMA).

8. The method of claim 7 wherein the number of said polyelectrolyte layers is greater than six.

9. The method of claim 8 wherein said polyelectrolytes are chemically crosslinked with itself, other said polyelectrolytes comprising said buffer film, and/or said substrate to increase stability, durability, and adhesion of said buffer film.

10. The method of claim 9 wherein said protective film consists of a multilayer comprising alternating layers of oppositely-charged cationic and anionic polyelectrolytes layers and enzymes capable of degrading chemical agents and arranged in layer fashion such that oppositely-charged adjacent layers electrostatically binding the multilayer together are formed.

11. The method of claim 10 wherein said cationic polyelectrolyte components are selected from the group consisting of protonated polyethylenimine (PEI), polyallylamine hydrochloride (PAH), and polydiallyldimethylammonium chloride (PDDA) and wherein said anionic polyelectrolyte components are selected from the group consisting of alkali metal salts of polyvinyl sulfate (PVS), polystyrenesulfonate (PSS), polyacrylate (PAA), and polymethacrylate (PMMA) and wherein said charged enzymes are selected from the group consisting of organophosphorous hydrolases (OPHs).

12. The method of claim 11 having the general repetitive layer structure (PEI/OPH/PEI/PSS)ₓ, wherein x is an integer greater than or equal to one, and wherein the order of deposition on said substrate is PEI, OPH, PEI, PSS.

13. The method of claim 12 further including the step chemically crosslinking the polyelectrolyte with itself and/or other polyelectrolytes comprising said protective film to increase stability and durability of said protective film.

14. The method of claim 13 further including the step of coating the polyelectrolyte layer with a charged polyelectrolyte layer bearing one selected from the group consisting of n-alkylpyridinium, n-alkylquaternary ammonium, and n-alkylquaternary phosphonium functional groups
and wherein the polyelectrolyte layer has at least one n-alkyl chain of about 4-18 carbon atoms in length, wherein the polyelectrolyte layer has a ligand functional group selected from the group consisting of humates, phosphatidylcholines, and β-hydroxyquinoline derivatives, or mixtures of said functional groups, so as to provide protection against both chemical and biological agents using a single protective film.

15. The method of claim 14 further including the step of coating the polyelectrolyte layer with a cationic polyelectrolyte selected from the group consisting of PEI and PAH, and
an organosiloxane film having
n-alkylpyridinium, n-alkylquaternary ammonium, or n-alkylquaternary phosphonium functional groups, and a ligand functional group selected from the group consisting of humates, phosphatidylcholines, and β-hydroxyquinoline derivatives, or mixtures of said functional groups thereof,
so as to provide protection against both chemical and biological agents using a single protective film.

16. The method of claim 15 further including the step of coating the protective film with a capping layer including a terminal outermost layer and
wherein said terminal outermost layer comprises
a N-[3-trimethoxysilyl)propyl]ethylenediamine capping agent which is capable of self-crosslinking polymerization to form a protective covalent network over said protective film so as to provide protection against both chemical and biological agents using a single protective film.
